(19) United States
(12) Patent Application Publication
(10) Pub. No.: US 2019/0224240 A1
(43) Pub. Date: Jul. 25, 2019

(54) CELL THERAPY FOR THE TREATMENT OF NON-ISCHEMIC HEART FAILURE

(71) Applicant: CardioCell, LLC, San Diego, CA (US)

(72) Inventor: Javed Butler, Atlanta, GA (US)

(73) Assignee: CardioCell, LLC, San Diego, CA (US)

(21) Appl. No.: 16/069,065

(22) PCT Filed: Jan. 11, 2017
(86) PCT No.: PCT/US2017/013064

§ 371 Date: Mar. 27, 2019

Related U.S. Application Data
(60) Provisional application No. 62/380,386, filed on Aug. 27, 2016.
(60) Provisional application No. 62/277,145, filed on Jan. 11, 2016.

(51) Int. Cl. A61K 35/28; A61K 9/00; A61P 9/00

(52) U.S. Cl. CPC A61K 35/28; C12N 5/0663; A61P 9/00; A61K 9/0019

(57) ABSTRACT

The invention provides methods of treating non-ischemic heart failure through the intravenous administration of mesenchymal stem cells. The methods can be practiced with ischemic tolerant mesenchymal stem cells and chronic ischemic tolerant mesenchymal stem cells. The methods can improve cardiac function in treated patients, including improvements in six-minute walk performance. The methods provide a therapeutic outcome without intracardial injection of cell therapy thereby avoiding further injury to an already compromised heart.

### CROSS-REFERENCE TO RELATED APPLICATIONS

This application claims the benefit of International Application No. PCT/US2017/013064 filed Jan. 11, 2017 which claims priority to U.S. Provisional Application No. 62/380,386 filed Aug. 27, 2016 and U.S. Provisional Application No. 62/277,145 filed Jan. 11, 2016. The entire contents of these applications are incorporated herein by reference in their entirety for all purposes.

### FIELD OF THE INVENTION

The invention generally relates to cell therapy for the treatment of heart failure. More particularly, the invention relates to the systemic administration of mesenchymal stem cells in the treatment of non-ischemic heart failure.

### DESCRIPTION OF RELATED ART

Myocardial dysfunction is fundamental to heart failure pathophysiology and mediates poor patient outcomes. Existing therapies targeting maladaptive neurohormones may partially recover left ventricular function. The neutral results from several recent large trials of neurohormonal blocking agents in hospitalized heart failure (Massie 2010; Konstam 2007; Gheorghiade 2013) however, support a shift in focus toward treatments that directly target the failing heart (Bayeva 2013; Schelbert 2014; Wong 2013). Stem cell therapy represents an emerging investigational treatment for heart failure, but several preclinical and clinical studies in ischemic and non-ischemic cardiomyopathy using various cell preparations have thus far failed to conclusively demonstrate efficacy (Bolli 2011; Nowbar 2014; Sanganalmath 2013). In addition, current cell-based therapies are directed to the intracardial administration of cells. Such therapies have the disadvantage of presenting complications, including injury to the coronary artery and haemorrhage within the pericardium, or tamponade (constriction of the cardiac blood vessels).

Despite contemporary therapy, outcomes for many non-ischemic heart failure patients remain poor. Promising results with many early-phase drug studies have failed to be replicated in larger late-phase trials assessing clinical outcomes. What is needed in the art therefore is a non-invasive cell-based therapy for treating heart failure with demonstrated efficacy.

### BRIEF SUMMARY OF THE INVENTION

The present invention overcomes existing shortcomings in the treatment of heart failure by providing an efficacious, non-invasive cell therapy for treating patients with dysfunctional myocardium resulting from a non-ischemic etiology.

It is therefore an object of the invention to provide a method of treating non-ischemic heart failure in a patient in need thereof, wherein the method comprises administering to the patient mesenchymal stem cells, wherein administering the mesenchymal stem cells to the patient treats non-ischemic heart failure in the patient.

In an aspect of the invention, the mesenchymal stem cells are administered to the patient systemically.

In a further aspect of the invention, the mesenchymal stem cells are administered to the patient intravenously.

In a further aspect of the invention, the mesenchymal stem cells are ischemic tolerant mesenchymal stem cells.

In a further aspect of the invention, the mesenchymal stem cells are chronic ischemic tolerant mesenchymal stem cells.

In a further aspect of the invention, the patient is a human.

In a further aspect of the invention, the mesenchymal stem cells are allogeneic mesenchymal stem cells.

In a further aspect of the invention, the mesenchymal stem cells are bone marrow mesenchymal stem cells.

In a further aspect of the invention, the mesenchymal stem cells express CD105, CD73 and CD90.

In a further aspect of the invention, the patient has NYHA class II or NYHA class III heart failure.

In a further aspect of the invention, the patient has dysfunctional viable myocardium without scarring.

In a further aspect of the invention, the patient has a reduced left ventricular ejection fraction.

In a further aspect of the invention, administering the mesenchymal stem cells to the patient improves cardiac function in the patient compared to the patient's cardiac function prior to administering the mesenchymal stem cells.

In a further aspect of the invention, administering the mesenchymal stem cells to the patient improves the patient's Kansas City Cardiomyopathy Questionnaire (KCCQ) score.

In a further aspect of the invention, administering the mesenchymal stem cells to the patient improves six minute walk performance in the patient.

A further object of the invention is to provide a method of improving cardiac function in a patient in need thereof, wherein the method comprises administering to the patient mesenchymal stem cells, wherein administering the mesenchymal stem cells to the patient improves cardiac function in the patient.

In an aspect of the invention, the mesenchymal stem cells are administered to the patient systemically.

In a further aspect of the invention, the mesenchymal stem cells are administered to the patient intravenously.

In a further aspect of the invention, the mesenchymal stem cells are ischemic tolerant mesenchymal stem cells.

In a further aspect of the invention, the mesenchymal stem cells are chronic ischemic tolerant mesenchymal stem cells.

In a further aspect of the invention, the mesenchymal stem cells are exposed to low oxygen.

In a further aspect of the invention, the patient is a human.

In a further aspect of the invention, the mesenchymal stem cells are allogeneic mesenchymal stem cells.

In a further aspect of the invention, the mesenchymal stem cells are bone marrow mesenchymal stem cells.

In a further aspect of the invention, the mesenchymal stem cells are purified.

In a further aspect of the invention, the mesenchymal stem cells express CD105, CD73 and CD90.

In a further aspect of the invention, the patient has non-ischemic heart failure.

In a further aspect of the invention, the patient has NYHA class II or NYHA class III heart failure.

In a further aspect of the invention, the patient has dysfunctional viable myocardium without scarring.

In a further aspect of the invention, the patient has a reduced left ventricular ejection fraction.

In a further aspect of the invention, administering the mesenchymal stem cells to the patient improves the patient's Kansas City Cardiomyopathy Questionnaire (KCCQ).

In a further aspect of the invention, administering the mesenchymal stem cells to the patient improves six-minute walk performance in the patient.

These and other objects of the invention will be apparent to one skilled in the art in view of the present description.

### BRIEF DESCRIPTION OF THE DRAWINGS

FIG. 1 shows HIF-1 expression of chronic itMSC.

FIG. 2 shows VEGF expression of chronic itMSC.

FIG. 3 shows the migratory capacity of chronic itMSC.

FIG. 4 shows heart function in human patients 90 days after intravenous administration of chronic itMSC.

FIG. 5 shows MRI data as a percentage of change from baseline for treated (E) and placebo (P) human patients 90 days after intravenous administration of chronic itMSC.

FIG. 6 shows cardiac function in human patients 90 days after intravenous administration of chronic itMSC.

FIG. 7 shows quantitative improvement of cardiac function in human patients 90 days after intravenous administration of chronic itMSC.

FIG. 8 shows Kansas City Cardiomyopathy Questionnaire (KCCQ) scores in human patients 90 days after intravenous administration of chronic itMSC.

FIG. 9 shows New York Heart Association (NYHA) functional classifications of human patients 90 days after intravenous administration of chronic itMSC.

FIG. 10 summarizes the study design for the extended study of the administration of chronic itMSC in the treatment of non-ischemic cardiomyopathy in human patients.

FIG. 11 shows the functional improvement in human patients following the administration of chronic itMSC.

FIG. 12 shows the correlation of the reduction in NK cells from baseline to 90 days and the magnitude of improvement in LVEF in human patients following the administration of chronic itMSC.

### DEFINITIONS

The terms “mesenchymal stem cell” and “MSC” are used interchangeably herein to refer to multipotent stromal cells that can differentiate into a variety of cell types of the mesenchyme cell lineage. Mesenchymal stem cells include ischemic tolerant mesenchymal stem cells, chronic ischemic tolerant mesenchymal stem cells, and mesenchymal stem cells that have not been exposed to hypoxic conditions in vitro or ex vivo.

The terms “ischemic tolerant mesenchymal stem cells” and “itMSC” are used interchangeably herein to refer to mesenchymal stem cells that have been exposed to low oxygen conditions in vitro or ex vivo. Such exposure includes, but is not limited to, preconditioning, culturing and/or growing mesenchymal stem cells under low oxygen conditions.

The terms “chronic ischemic tolerant mesenchymal stem cells” and “chronic itMSC” are used interchangeably herein to refer to ischemic tolerant mesenchymal stem cells that have been maintained exclusively under low oxygen conditions in vitro or ex vivo.

The term “treatment” as used herein refers to amelioration or reduction of a symptom or a condition affecting an organism, such as a mammal, including but not limited to, humans.

The terms “heart failure” and “cardiomyopathy” are used interchangeably herein to refer to a condition that occurs when a problem with the structure or function of the heart impairs its ability to supply sufficient blood flow to meet the physiological requirements of the body.

The terms “non-ischemic heart failure” and “non-ischemic cardiomyopathy” are used interchangeably herein to refer to heart failure that results from any cause or condition that is not associated with the interruption of blood flow to the heart, such as interruption of blood flow to the heart resulting from myocardial infarction or coronary artery disease, for example.

The terms “low oxygen conditions,” “low oxygen,” “reduced oxygen tension,” “hypoxic,” and “hypoxia” are used interchangeably to refer to any oxygen concentration that is less than atmospheric oxygen. Low oxygen conditions include, but are not limited to, an oxygen concentration at or about 20%, 19%, 18%, 17%, 16%, 15%, 14%, 13%, 12%, 11%, 10%, 9%, 8%, 7%, 6%, 5%, 4%, 3%, 2%, or 1%. Low oxygen conditions include 5% oxygen.

The terms “about” and “approximately” mean within 20%, more preferably within 10%, and most preferably within 5% of a given value or range. Alternatively, especially in biological systems, the term “about” means within about a log (i.e., an order of magnitude) preferably within a factor of two of a given value.

### DETAILED DESCRIPTION

The invention generally relates to a cell-based therapy for the treatment of non-ischemic heart failure. More particularly, the invention relates to treating non-ischemic heart failure through the administration of mesenchymal stem cells.

In one aspect, the invention provides a method for treating non-ischemic heart failure in a patient in need thereof, wherein the method comprises administering to the patient mesenchymal stem cells.

Mesenchymal stem cells for use with the invention can be purified or non-purified. Mesenchymal stem cells for use with the invention include, but are in no way limited to, those described in the following references, the disclosures of which are incorporated herein by reference in their entirety for all purposes: U.S. Pat. Nos. 5,215,927; 5,225,353; 5,262,334; 5,240,856; 5,486,359; 5,759,793; 5,827,735; 5,811,094; 5,736,396; 5,837,539; 5,837,670; 5,827,740; 6,087,113; 6,387,367; 7,060,494; 8,790,638; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705. Unless otherwise indicated by the context in which the term is used, “mesenchymal stem cells,” or “MSC,” as used herein includes, normoxically grown MSC, itMSC and chronic itMSC.

Mesenchymal stem cells for use with the invention can be ischemic tolerant mesenchymal stem cells (itMSC). The itMSC can be mesenchymal stem cells that have been pre-conditioned under hypoxic conditions. The itMSC can be mesenchymal stem cells that are preconditioned by exposure to hypoxic conditions, in vitro or ex vivo, for 2, 4, 6, 8, 10, 12, 14, 16, 18, 20, 22, 23, or 24 hours, as well as any time intervening these specifically listed times. The itMSC can be mesenchymal stem cells that are preconditioned by exposure to hypoxic conditions, in vitro or ex vivo, without permitting the cells to divide in vitro or ex vivo. The itMSC can be exposed to normoxic conditions before, after, or both before and after the itMSC are preconditioned under hypoxic conditions. The itMSC can be mesenchymal stem cells that have been expanded under hypoxic conditions. The itMSC can undergo one or more passages under hypoxic conditions. The itMSC can be mesenchymal stem cells that have undergone 1, 2, 3, 4, 5, 6, 7, 8, 9, 10, 11, 12, 13, 14, 15, 16, 17, 18, 19, 20, 21, 22, 23, 24, 25, or more passages under hypoxic conditions. The itMSC can be mesenchymal stem cells that have undergone five passages under hypoxic conditions. The itMSC can be exposed to normoxic conditions before, after, or both before and after the itMSC are expanded under hypoxic conditions. The itMSC can express CD73, CD90, CD105, and CD166. The itMSC can express CD73, CD90, CD105, and CD166, but lack the expression of HLA-DR. The itMSC administered to the patient can be purified and free of any other cell type or other agent that has a biological effect in the patient.

In some aspects of the invention, itMSC for use with the invention are chronic itMSC. Chronic itMSC are itMSCs that have been maintained exclusively under low oxygen conditions in vitro or ex vivo. The chronic itMSC can be expanded under low oxygen conditions for 1, 2, 3, 4, 5, 6, 7, 8, 9, 10, 11, 12, 13, 14, 15, 16, 17, 18, 19, 20, 21, 22, 23, 24, 25, or more passages. The chronic itMSC can be expanded under 5% oxygen. The chronic itMSC can be grown exclusively under low oxygen conditions beginning with a primary culture of cells (p0), that is subsequently grown for one or more passages under low oxygen conditions. The chronic itMSC can be grown exclusively under low oxygen conditions beginning with a primary culture of cells (p0), that is subsequently grown for 1, 2, 3, 4, 5, 6, 7, 8, 9, 10, 12, 13, 14, 15, 16, 17, 18, 19, 20, 21, 22, 23, 24, 25, or more passages under low oxygen conditions. Chronic itMSC can be grown under low oxygen conditions beginning with a primary culture of cells that is then grown for 4 or 5 passages under low oxygen conditions. Chronic itMSC can be maintained under low oxygen conditions from the time of collection from a donor of the mesenchymal stem cells, during expansion of the mesenchymal stem cells, and through the process of preparing the mesenchymal stem cells for administration to a patient. Accordingly, chronic itMSC may be combined with a pharmaceutical carrier to produce a composition having a low oxygen concentration that is ready for administration to a patient. Chronic itMSCs can be mesenchymal stem cells that have been extracted from a donor, purified or enriched, and administered to the patient, all under low oxygen conditions. Chronic itMSCs can be mesenchymal stem cells that have been extracted from a donor, purified or enriched, expanded, and administered to the patient, all under low oxygen conditions. Chronic itMSCs can be mesenchymal stem cells that have been extracted from a donor, purified or enriched, expanded, cryogenically preserved, thawed, and administered to the patient, all under low oxygen conditions. Chronic itMSCs can be mesenchymal stem cells that have been extracted from a donor, selectively expanded, and administered to the patient, all under low oxygen conditions. Chronic itMSCs can be mesenchymal stem cells that have been extracted from a donor, selectively expanded, cryogenically preserved, thawed, and administered to the patient, all under low oxygen conditions. In a non-limiting embodiment of the invention, chronic itMSCs are mesenchymal stem cells that have been extracted from a patient, purified or enriched, expanded for five passages, cryogenically preserved, thawed, and administered to the patient, all under 5% oxygen. Chronic itMSC can express CD73, CD90, CD105, and CD166. Chronic itMSC can express CD73, CD90, CD105, and CD166, but lack the expression of HLA-DR. Chronic itMSC administered to the patient can be purified and free of any other cell type or other agent that has a biological effect in the patient.

Mesenchymal stem cells for use with the invention can be cultured in serum or under serum-free conditions. The term “low serum conditions” refers to culturing cells in a culture medium comprising less than about 25% serum. Low serum conditions include, but are not limited to, culturing mesenchymal stem cells in a culture medium comprising 20% serum, 15% serum, 10% serum, 5% serum, or any amount of serum intervening these specifically referenced amounts. Low serum conditions include, but are not limited to, culturing mesenchymal stem cells in a culture medium comprising about 20% serum, about 15% serum, about 10% serum, or about 5% serum. The mesenchymal stem cells can be itMSC cultured under low serum conditions. The mesenchymal stem cells can be itMSC cultured in a culture medium comprising about 5% serum. The mesenchymal stem cells can be chronic itMSC cultured under low serum conditions. The mesenchymal stem cells can be chronic itMSC cultured in a culture medium comprising about 5% serum. The serum can be any serum suitable for conventional mammalian cell culture. The serum can be selected from the group consisting of bovine serum, mouse serum, rat serum, rabbit serum, goat serum, horse serum, human serum, and a combination thereof. The fetal bovine serum can be selected from the group consisting of newborn calf serum (from calves under 3 weeks old), cadet calf serum (<6 months old), calf serum (<12 months old), adult bovine serum (<12 months), donor bovine serum (<36 months old), and a combination thereof.

Mesenchymal stem cells for use with the invention can be obtained from any tissue source of capable of providing mesenchymal stem cells that produce a therapeutic effect in treating the patient's non-ischemic heart failure according to the methods disclosed herein. The tissue source can be selected from the group consisting of prenatal, postnatal, and a combination thereof. Tissues for deriving the mesenchymal stem cells can be selected from the group consisting of bone marrow, dermis, periosteum, synovium, peripheral blood, skin, hair root, dermal papilla, muscle, uterine endometrium, adipose, placenta, menstrual discharge, chorionic villus, amniotic fluid, umbilical cord blood, and a combination thereof. The mesenchymal stem cells can be bone marrow mesenchymal stem cells. The mesenchymal stem cells can be itMSC obtained from bone marrow. The mesenchymal stem cells can be allogeneic itMSC obtained from bone marrow. The mesenchymal stem cells can be chronic itMSC obtained from bone marrow. The mesenchymal stem cells can be allogeneic chronic itMSC obtained from bone marrow. The mesenchymal stem cells can be derived from bone marrow sources selected from the group consisting of flat bones, long bones, and a combination thereof. Flat bones for providing the bone marrow mesenchymal stem cells can be selected from the group consisting of the pelvis, sternum, cranium, ribs, vertebrae, scapulae, and a combination thereof. Long bones for providing the bone marrow mesenchymal stem cells can be selected from the group consisting of the femur and humerus. It is further contemplated that the mesenchymal stem cells can be differentiated from embryonic stem cells, induced pluripotent stem cells, or a combination thereof. The mesenchymal stem cells can be obtained from a clonal cell line of mesenchymal stem cells.

Mesenchymal stem cells for practicing the invention may be obtained from a donor source selected from the group consisting of: allogeneic, syngeneic, xenogeneic or a combination thereof. The mesenchymal stem cells can be obtained from a human donor source. The mesenchymal stem cells can be human itMSC. The mesenchymal stem cells can be human chronic itMSC. The mesenchymal stem cells can be allogeneic itMSC. The mesenchymal stem cells can be allogeneic chronic itMSC. Allogeneic mesenchymal stem cells can be HLA matched with the patient that is to be treated. The mesenchymal stem cells can be obtained from healthy, young human donors that are between about 18 to 25 years old. The mesenchymal stem cells can be obtained from the bone marrow of healthy, young human donors that are between about 18 to 25 years old. The mesenchymal stem cells can be xenogeneic with respect to the patient. Xenogeneic mesenchymal stem cells can be obtained from sources selected from the group consisting of: bovine; equine; porcine; murine; capra (e.g. goats and sheep); and leporidae (e.g. rabbits and hares).

In an aspect of the invention, mesenchymal stem cells are administered to the patient systemically. The patient can be a mammal, preferably a human. The mesenchymal stem cells can be administered to the patient according to routes selected from intravenously, intraarterially, and a combination thereof. Intravenous administration can be by one or more peripheral IVs, one or more central IVs, one or more implantable ports, or a combination thereof. Intravenous administration can be by continuous infusion (e.g. drip), secondary IV, IV push, or a combination thereof. The mesenchymal stem cells can be administered by a route selected from the group consisting of: intracoronary; transendocardial; intramyocardial administration; and a combination thereof.

The mesenchymal stem cells can be combined with one or more pharmaceutical acceptable carriers for administration to the patient. As used herein, the term “pharmaceutically acceptable carrier” refers to reagents, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Suitable pharmaceutical carriers for use with the invention, include, but are not limited to, those described in Remington's Pharmaceutical Sciences, 19th Edition, Mack Publishing Co., Easton, Pa. 1995, the entire contents of which are incorporated herein by reference in their entirety for all purposes. The pharmaceutical carrier can be suitable for injection. Suitable pharmaceutical carriers for use with the invention can be selected from the group consisting of: saline; sterile water; phosphate buffered saline; Ringers solution; isotonic dextrose; sterile culture media; and a combination thereof. The pharmaceutically acceptable carrier can be an artificial substance suitable for administration to a patient.

The mesenchymal stem cells can be administered to the patient in an amount effective to treat non-ischemic heart failure in the patient. As used herein, the terms “effective amount,” and “amount effective to treat non-ischemic heart failure,” refer to any amount of mesenchymal stem cells the administration of which provides a therapeutic effect in addressing one or more of the underlying causes and/or symptoms of non-ischemic heart failure in the patient. An effective amount of mesenchymal stem cells can be between about 10-×10⁶ MSC and 1×10⁵ mesenchymal stem cells per kilogram of the patient's bodyweight. An effective amount of mesenchymal stem cells can be 1.5×10⁶ cells/kg body weight. The mesenchymal stem cells can be administered to the patient one, two, three, four, five, six, seven, eight, nine, ten or more times. The mesenchymal stem cells can be administered one or more times per day, one or more times per week, one or more times per month, or one or more times per year. One skilled in the art will appreciate that multiple administrations of the mesenchymal stem cells may be required if a lack of alleviation, or further deterioration of, the symptoms of the patient's heart failure is observed following one or more administrations of the stem cells.

In an aspect of the invention, ischemic tolerant mesenchymal stem cells are administered to a patient in need of treatment for non-ischemic heart failure. Patients in need of treatment for non-ischemic heart failure can have non-ischemic heart failure due to any disease or condition that does not result in the interruption of blood flow to the heart. The patient can be male or female. The patient in need of treatment can have no history of myocardial infarction and absent or non-obstructive coronary artery disease. The patient can have an absence of significant epicardial coronary artery disease. The patient can be identified as having absent or non-obstructive coronary artery disease by computed tomography or coronary angiography. The patient can have non-obstructive coronary artery disease wherein the patient has less than 30% left main stenosis or less than 50% stenosis of any major epicardial coronary artery. The patient can have no evidence of myocardial scarring. The patient can be identified as having no evidence of myocardial scarring by delayed-enhancement cardiac magnetic resonance. The patient can have dysfunctional, but still viable myocardium.

The patient for treatment according to the method of the invention can have non-ischemic heart failure resulting from any cardiomyopathy wherein the cause does not result from restricted blood flow to the heart. The patient having non-ischemic heart failure can have a condition selected from the group consisting of: dilated cardiomyopathy; restrictive cardiomyopathy; arrhythmogenic right ventricular dysplasia; hypertrophic cardiomyopathy; viral infection of the heart; viral hepatitis; diabetes; exposure to toxins such as heavy metals or chemotherapy; exposure to radiation therapy; amyloidosis; hematochromatosis; drug abuse such as methamphetamine and/or cocaine; alcoholism; sarcoidosis; and a combination thereof. The patient can have idiopathic non-ischemic heart failure.

The patient selected for treatment according to the invention can have one or more symptoms of heart failure. The patient can have a reduced left ventricular ejection fraction (LVEF). As used herein, the phrase “reduced left ventricular ejection fraction,” or “reduced LVEF,” refers to an ejection fraction that is about 40% or less. The reduced ejection fraction can be 40%, 35%, or 25%, as well as any amount that intervenes these specifically listed ejection fractions. A reduced LVEF in the patient can be determined based on a comparison to the patient's baseline LVEF. A reduced LVEF in the patient can be determined by a comparison to a normal LVEF that is derived from a population of healthy subjects. Such subjects can be comparable to the patient in at least one of age, weight, height, body mass index, lifestyle, activity level, and ethnicity. The patient can have abnormal wall motion as determined by echocardiography. The patient can have a reduced coronary function as determined by at least one of the New York Heart Association (NYHA), Kansas City Cardiomyopathy Questionnaire (KCCQ), and EuroHeart Failure Survey classification scores. The patient can have a NYHA classification score of II, III or IV. The patient can have symptoms of non-ischemic heart failure as determined by a six-minute walk assessment.

Treating a patient according to the invention can improve, or prevent further deterioration of, one or more symptoms of non-ischemic heart failure. Treatment can improve, or prevent further deterioration of, symptoms selected from the group consisting of: LVEF; abnormal wall motion; left ventricular end-systolic dimensions; left ventricular end-diastolic dimensions; systolic pulmonary artery pressures; NYHA classification scores; KCCQ classification scores; EuroHeart classification scores; six-minute walk performance; and a combination thereof. Treatment according to the invention can raise the patient's score according to one or more of the NYHA, KCCQ, and EuroHeart Failure Survey classifications. Treatment according to the invention can prevent further deterioration in the patient's score according to one or more of the NYHA, KCCQ, and EuroHeart Failure Survey classifications. In one non-limiting embodiment, treating the patient according to the invention improves the patient's six-minute walk performance.

The following examples are illustrative only and are not intended to limit the scope of the invention to the embodiments and limitations described in the examples.

### Example 1—Administration of Ischemic Tolerant Mesenchymal Stem Cells in the Treatment of Non-Ischemic Heart Failure in Human Patients

### Patients

The study population consisted of ambulatory patients with chronic non-ischemic heart failure with LVEF 40% or less, age at least 18 years old, and New York Heart Association (NYHA) class II/III symptoms. Patients have been receiving stable maximally tolerated doses of guideline-directed medical therapies for heart failure with reduced LVEF (at the discretion of the investigator) for at least 6 months before randomization. Patients had no evidence of replacement scarring on delayed-enhancement cardiac magnetic resonance (DE-CMR). Additional inclusion and exclusion criteria are shown in Tables 1 and 2.

TABLE 1
Selected inclusion criteria

≥18 years of age
LVEF ≤40% documented on echocardiogram within 6 months of randomization
Cine-CMR with LVEF ≤40% and no evidence of scarring on DE-CMR
Nonischemic heart failure cause, defined as no documented history of myocardial
infarction and absent or nonobstructive CAD on coronary angiography or
coronary CTA within 3 years of randomization
Nonobstructive coronary artery disease is defined as <30% left main stenosis or
<50% stenosis of any major epicardial coronary artery
Receiving stable maximally tolerated HF medical therapy (at the discretion of the
investigator) including ACEI, ARB, β-blockers, MRA for ≥6 months before
randomization
NYHA class II/III symptoms
ACEI, angiotensin-converting enzyme inhibitor;
ARB, angiotensin II receptor blocker;
CAD, coronary artery disease;
CMR, cardiac magnetic resonance;
CTA, computed tomography angiogram;
DE-CMR, delayed-enhancement CMR;
LVEF, left ventricular ejection fraction;
MRA, mineralocorticoid receptor antagonist;
NYHA, New York Heart Association.

TABLE 2
Selected exclusion criteria

Cardiac arrest, life-threatening arrhythmia, or stroke within 3 months of randomization
Cardiac surgery within 3 months of randomization or high likelihood of requiring
cardiac surgery during the study period
Current ICD, PPM, or CRT implantation, or planned implantation within 6 months of
infusion
Clinically significant uncorrected valve disease, hypertrophic or restrictive cardiomyopathy,
active myocarditis, uncontrolled hypertension, or complex congenital
heart disease
Treatment with parenteral inotropic agents within 1 month of randomization
Current left ventricular assist device, or planned implantation within 6 months of
infusion
Illness other than heart failure with life expectancy <1 year
Liver disease (ALT or AST >3× normal; alkaline phosphatase or bilirubin >2×
normal)
Renal disease (eGFR <30 ml/min by MDRD formula)
Hematologic disease (leukocytosis >10 × 10⁹/l or hemoglobin <9 g/dl)
ALT, alanine aminotransferase;
AST, aspartate aminotransferase;
CRT, cardiac resynchronization therapy;
eGFR, estimated glomerular filtration rate;
ICD, implantable cardioverter-delibrillator;
MDRD, Modification of Diet in Renal Disease;
PPM, permanent pacemaker.

### Study Therapy

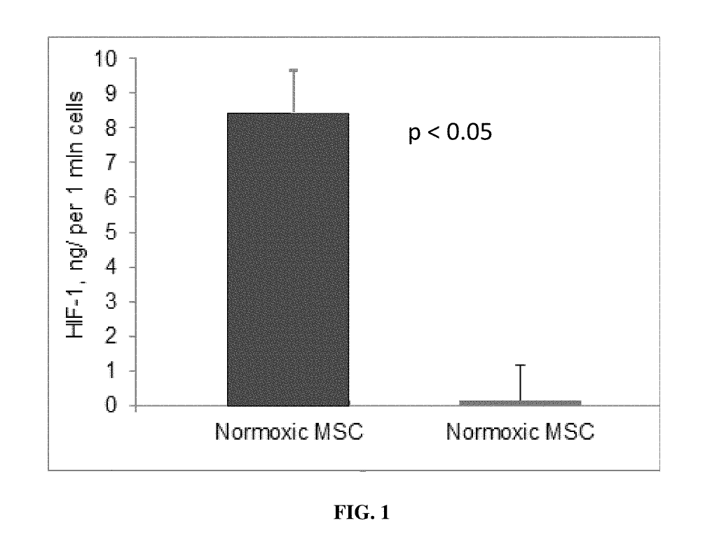

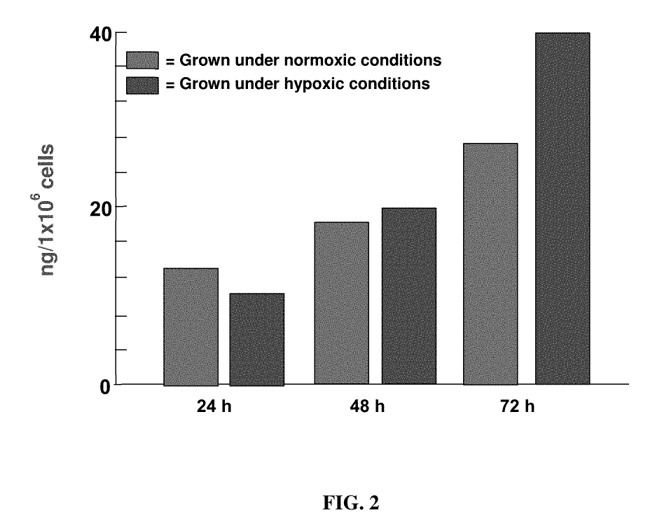

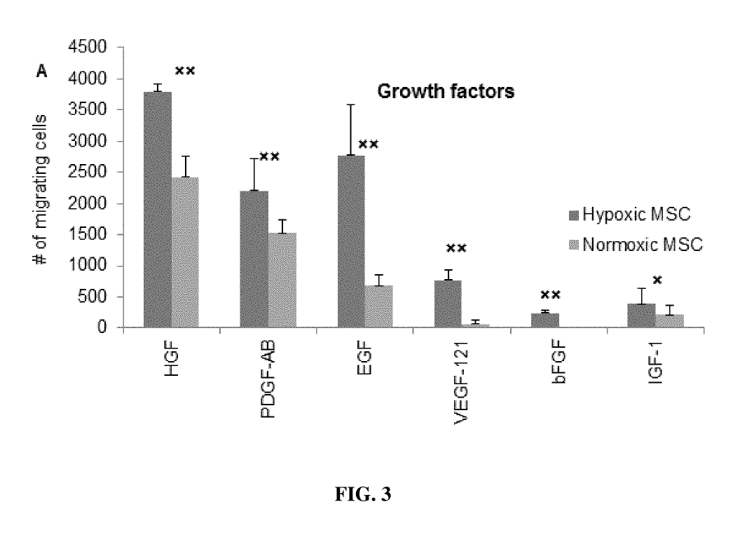

The experimental therapy was an intravenous (IV) infusion of chronic ischemic tolerant human donor allogeneic MSCs (itMSCs), dosed at 1.5 million cells per kilogram. All cells expressed CD105, CD73, and CD90 surface markers and were extracted from the bone marrow of healthy 18-25 year old human volunteers. From the moment of extraction, cells were grown under hypoxic conditions (5% oxygen and expanded up to passage 5). MSCs grown under these conditions demonstrated enhanced expression of HIF-1 (FIG. 1) and VEGF (FIG. 2) and enhanced migratory capacity (FIG. 3).

For cryopreservation, harvested cells were suspended in Cryostar CS10 freezing medium (BioLife Solutions, Bothell, Wash., USA), frozen in a controlled rate freezer, and stored in the vapor phase of liquid nitrogen. Within 8 hours before infusion, cells were thawed at the site pharmacy and re-suspended in Lactated Ringer's solution at a concentration of 1×10⁶ cells/ml. The placebo therapy was an IV infusion of Lactated Ringer's solution at a volume of 1 ml/kg. The dose of itMSCs was based on a prior study of these cells in human patients with ischemic stroke (Clinical-Trials.gov identifier: NCT01297413). In that study, doses as high as 1.5 million cells per kilogram were shown to be well tolerated.

### Study Protocol

This was a single-blind, placebo-controlled, crossover, multicenter, randomized study with blinded endpoints to evaluate the safety and efficacy of ischemic tolerant MSCs when added to standard therapy in non-ischemic heart failure patients with LVEF 40% or less. Participants received itMSC therapy or placebo according to their assigned study group. At 90 days' post-initial infusion, the two groups will receive the alternative treatment in the crossover phase. Thus, at 180 days each patient will receive two infusions (one MSC infusion and one placebo infusion) during the study, with subsequent cardiac structure and function assessment at both 90 and 180 days' post-initial infusion, or at time of early termination. Complete examinations were used to assess the safety and efficacy endpoints during the follow-up period. Transthoracic echocardiograms were performed at screening and day 90 post-initial infusion. CMR was performed at baseline and day 90 post-initial infusion.

### Study Endpoints

The primary endpoint is the safety assessment, which was conducted at baseline and day 90 post-initial infusion. Data regarding the following was collected: procedural complications, vital signs, changes in heart failure medications, clinical arrhythmias with Holter monitoring, laboratory tests (including complete blood counts, comprehensive chemistry panels with liver function tests, troponin I, creatinine kinase), electrocardiogram, all-cause mortality, all-cause hospital admission, and need for heart failure co-intervention. Additionally, pulmonary function tests were performed at baseline and day 90.

The secondary endpoint is the change in LVEF as measured by CMR. Change in LVEF was evaluated during two intervals: between baseline and day 90 post-initial infusion. Exploratory endpoints will include measures of cardiac function using transthoracic echocardiography with Simpson's summation-of-disks method and speckle tracking at day 90 post-initial infusion. Details of exploratory endpoints are displayed in Table 3.

TABLE 3
Exploratory endpoints

Changes between baseline and day 90 post-initial infusion. After the
crossover phase, all patients will be evaluated for changes from the
new baseline (day 90 after initial phase) to day 90 after new infusion
CMR endpoints:
LV end-systolic volume index
Wall motion segmental score
LV end-systolic and end-diastolic dimensions
Extracellular volume fraction measured by T1-mapping
Transthoracic echocardiogram endpoints:
Changes in LV mechanics by speckle tracking (longitudinal and
circumferential strain and strain rate)
Estimated systolic pulmonary artery pressures
Changes in mitral regurgitation severity
Changes between baseline and days 30, 60, and 90 post-initial infusion.
After the crossover phase, all patients will be evaluated for changes from
the new baseline (day 90 after initial phase) to days 30 and 90 after new
infusion
Exercise capacity (6 · min walk distance)
Health status (NYHA class, Kansas City Cardiomyopathy Questionaire)
Heart failure status (clinical congestion score)
Biomarkers (BNP, troponin, FGF, VEGF)
BNP, B-type natriuretic peptide;
CMR, cardiac magnetic resonance;
FGF, fibroblast growth factor;
LV, left ventricular;
NYHA, New York Heart Association;
VEGF, vascular endothelial growth factor.

### Immunologic Safety

Immunologic safety of the study therapy was addressed in two different ways. First, lot release specifications for the cell therapy exclude lymphoid cells such as CD19 (B cells), CD45 (T cells), CD 14 (macrophages/dendritic cells), which may either activate patient immune response via antigen presenting cells or produce graft-versus-host reaction. Second, the cells were required to show very low (<2% of total cell population) expression of HLA-DR molecule, which is required to activate T-helper cells after binding to immunogenic peptides. The success of these two methods is confirmed by an excellent safety profile with these MSCs in preclinical toxicity studies and phase 1 human clinical trials. For further assessment, in the present study, IgA, IgE, IgG, and IgM levels and the lymphocyte proliferation panel was evaluated at baseline and days 30, 60, and 90, or early termination.

### Statistical Considerations

Statistical analyses were descriptive and included conventional summary statistics. No substitutions were made for missing data and analyses were based on available data only. Given the pilot nature of the study, the sample size was not determined by statistical power considerations, but was considered appropriate for an early-phase study. An independent Data Safety and Monitoring Board consisting of two cardiologists, one oncologist, and one statistician conducted ongoing review of the nature, frequency, and severity of the safety data.

### DISCUSSION

This pilot investigation studied the administration of ischemic tolerant MSCs in the treatment of non-ischemic heart failure by focusing on alignment of three key study design elements: the use of the optimal formulation of MSCs, the inclusion of the heart failure phenotype hypothesized as most likely to respond to therapy, and application of the appropriate imaging modality for evaluating structural and functional cardiac recovery.

The wide heterogeneity inherent to the heart failure population has become increasingly recognized as a key consideration for therapy development. A major contributor to the mostly disappointing results of recent heart failure trials relates to failure to match the study agent with the subset of patients most likely to benefit (Vaduganathan 2013; Greene 2014). Accordingly, the use of a ‘mechanistic translational phase’ in drug development has been proposed to facilitate improved understanding of the therapy's effect on the heart in a small homogenous population before progressing to a large phase 3 trial (Gheorghiade 2011). The goal of such a mechanistic study is not to elucidate cellular and subcellular mechanisms per se, but rather to explore effects of the therapy on laboratory and imaging markers of overall cardiac function and remodeling (e.g. natriuretic peptides, transthoracic echocardiography, and CMR parameters). The test cohort constituted a patient phenotype rigorously defined by multiple criteria including LVEF, heart failure cause, extent of coronary artery disease, delayed enhancement on CMR, cardiac structural abnormalities, comorbidities, and the like.

Rationale for using cardiac magnetic resonance parameters for study enrollment criteria and evaluation of cardiac recovery

The present study pre-specified comprehensive CMR assessment before enrollment and patients with evidence of replacement scarring were excluded. Delayed-enhancement CMR has emerged as the reference standard technique for imaging myocardial scar (Wagner 2003; Kim 2008; Kim 2000). Thus, serial CMR imaging was integrated into the present study protocol.

### Results

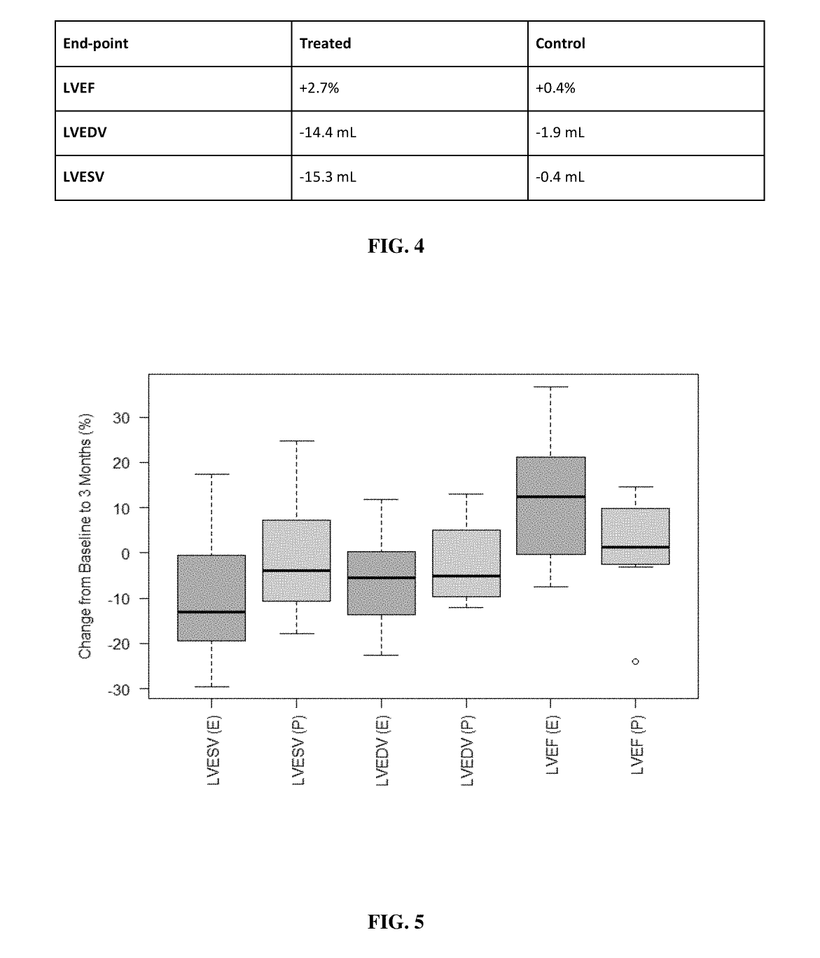

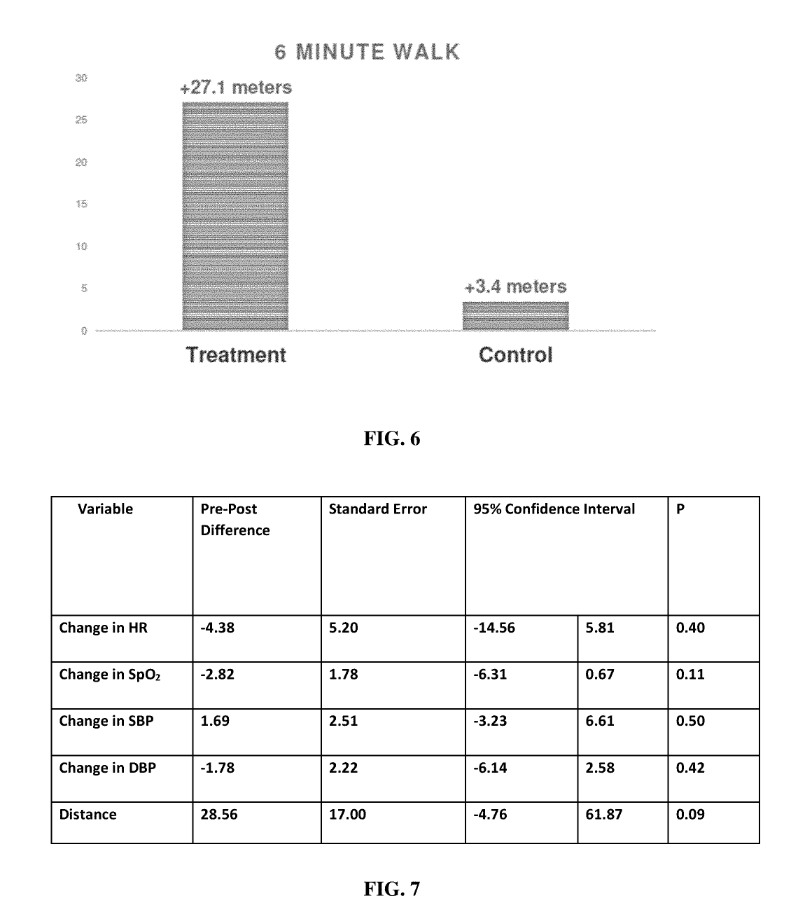

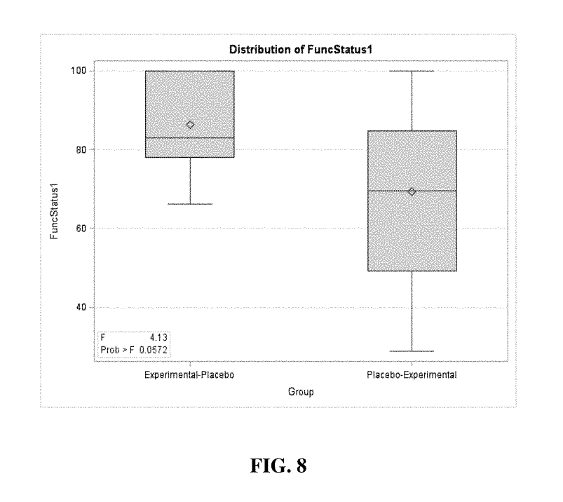

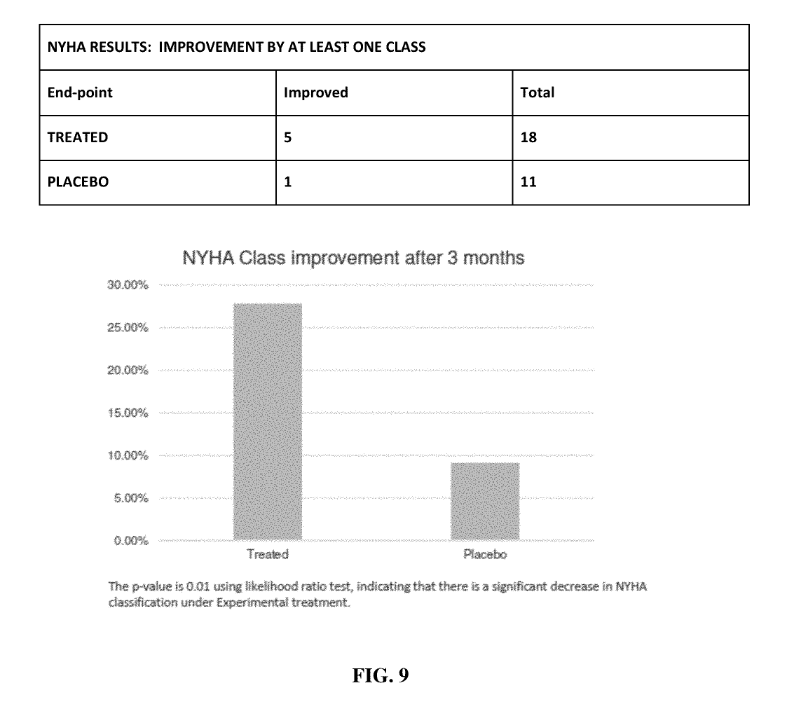

90 days after infusion with itMSC, treated patients showed: improved LVEF, LVEDV and LVESV (FIGS. 4 and 5); improvement in cardiac function as demonstrated by a six-minute walk (FIGS. 6 and 7); improved Kansas City Cardiomyopathy Questionnaire (KCCQ) scores (FIG. 8); and improved New York Heart Association (NYHA) functional classifications (FIG. 9).

### Example 2—Extended Study of the Administration of Ischemic Tolerant Mesenchymal Stem Cells in the Treatment of Non-Ischemic Heart Failure in Human Patients

### Background

Despite available therapies, patients with heart failure (HF) with reduced ejection fraction (HFrEF) continue to experience high rates of mortality and hospitalization (Ambrosy 2014). Accordingly, there has been increasing focus on alternative treatment strategies, including stem cell therapy (Kelkar 2015; Gheorghiade 2016). Despite intermittent positive signals in phase I and phase II trials, major reviews of available stem cell data have failed to show definitive benefit in HF (Sanganalmath 2013). However, virtually all published studies were centered on the concept that therapeutic efficacy depends on injected stem cells engrafting in the myocardium and either transdifferentiating into healthy cardiac myocytes or stimulating resident cardiac stem cells to expand and organize into functional myocardium (Narita 2015). Since the intravenous delivery of stem cells results in very low cardiac engraftment, existing clinical studies have been largely limited to the direct delivery of cells to the myocardium via intracoronary, trans endocardial, or intramyocardial methods.

Previous heart failure studies with interventions like beta-blockers and cardiac resynchronization therapy suggest that patients with non-ischemic cardiomyopathy generally demonstrate more robust response to therapy, potentially related to the more likely presence of dysfunctional but viable myocardium (Bayeva 2014; Cleland 2003; Barsheshet 2011; Wilcox 2015).

The present study is a phase II-a randomized trial designed to assess the safety, as well as cardiac and systemic effects, of intravenously administered itMSCs in human patients with chronic non-ischemic cardiomyopathy. The MSCs grown under chronic hypoxic conditions offer higher degrees of cytokine production and are relatively immune-privileged (Maslov 2013). The utilization of delayed-enhancement cardiac magnetic resonance (DE-CMR) imaging to better define patients with non-ischemic cardiomyopathy with absent myocardial scar increases sensitivity in detecting structural and functional cardiac responses to treatment (Kim 2000). To our knowledge, this trial represents the first published experience with intravenously administered chronic itMSCs in patients with any type of chronic cardiomyopathy.

This study was a single-blind, placebo-controlled, crossover, multicenter, randomized phase II-a trial with blinded endpoint assessment of patients with non-ischemic cardiomyopathy with left ventricular ejection fraction (LVEF)≤40% and absence of scar on cardiac magnetic resonance (CMR) imaging. Patients were randomized to 1.5×10⁶ intravenously administered chronic itMSCs or placebo; at 90 days each group received the alternative treatment. Primary endpoint was safety. Efficacy endpoints included changes in cardiac function, functional capacity, and health status.

Twenty-two human patients were randomized to chronic itMSC (n=10) and placebo (n=12) at baseline. After crossover, data were available for 22 chronic itMSC patients. Compared to placebo, chronic itMSC therapy increased 6-minute walk distance (+36.47 m, 95% CI 5.98-66.97, p=0.02), and improved Kansas City Cardiomyopathy clinical summary (+5.22, 95% CI 0.70-9.74, p=0.02) and functional status scores (+5.65, 95% CI −0.11-11.41, p=0.06). Data also demonstrated MSC-induced immunomodulatory effects, the magnitude of which correlated with the magnitude of improved LVEF. Among patients with non-ischemic cardiomyopathy, chronic itMSC therapy was safe and associated with improvements in health status and functional capacity.

### Methods

Eligible patients were ambulatory with chronic non-ischemic cardiomyopathy with LVEF ≤40% on baseline CMR, age ≥18 years old, and New York Heart Association (NYHA) class II/III symptoms. Non-ischemic cardiomyopathy was defined by: 1) no history of myocardial infarction; 2) absent or non-obstructive coronary artery disease (CAD) on invasive or computed tomography (CT) coronary angiography within 3 years prior to randomization, and 3) no evidence of scarring on DE-CMR. Patients were receiving stable maximally tolerated guideline-directed medical therapy for HFrEF for ≥six months prior to randomization. Due to the use of CMR, patients with current or planned implantation of an implantable-cardioverter defibrillator, permanent pacemaker, or cardiac resynchronization therapy devices were excluded, as were patients treated with inotropic agents within 1 month of randomization and patients with severe valvular, renal (estimated glomerular filtration rate <30 mL/min), or liver disease (alanine transaminase or aspartate transaminase >3× normal, alkaline phosphatase or bilirubin >2× normal). Patients who met eligibility criteria were blinded to treatment allocation and randomized 1:1 to receive intravenous chronic itMSC therapy or placebo. At 90 days' post-initial infusion, the two groups received the alternative treatment in the cross-over phase, thus resulting in each patient receiving two infusions (i.e., one chronic itMSC, one placebo) during the study. For reference, the two groups were labeled by the order in which they received treatment; placebo-chronic itMSC and chronic itMSC-placebo. Additionally, the term ‘control’ pertains to data from baseline to 90 days among the placebo-chronic itMSC group. The ‘chronic itMSC group’ refers to data from baseline to 90 days among the chronic itMSC-placebo group, as well as data from 90 days to 180 days among the placebo-chronic itMSC group.

The investigational therapy was an intravenous infusion of human donor allogeneic chronic itMSCs dosed at 1.5 million cells/kg. All cells expressed CD105, CD73, and CD90 surface markers and were extracted from the bone marrow of a young healthy human volunteer. Cells were grown under hypoxic conditions from the moment of extraction for 5 passages. For purposes of cryopreservation, cells were suspended in Cryostar CS10 freezing medium (BioLife Solutions, Bothell, Wash., USA), frozen in a freezer at a controlled rate, and stored in the vapor phase of liquid nitrogen. Within eight hours prior to patient infusion, cells were thawed within the pharmacy of the local study site and suspended in Lactated Ringer's solution at a concentration of 1×10⁶ cells/mL. The placebo therapy was an intravenous infusion of Lactate Ringer's solution at a volume of 1 mL/kg.

The primary safety endpoints were assessed at days 30, 60, 90, 120, 150, 180, 270, and 450 post-initial infusion. At each time point, all-cause mortality, all-cause hospitalization, and adverse events (including severity of event and perceived relationship to the study products as related or not related) were reported by study investigators. Vital signs, 12-lead electrocardiograms, and laboratory tests (including complete blood counts, comprehensive chemistry panels, troponin I, and creatinine kinase) were collected at each time point. 24-hour Holter monitoring was done at all time points through 270 days' post-initial infusion. An independent data and safety monitoring board monitored patient safety during the trial.

Multiple pre-specified secondary efficacy endpoints included CMR changes in LVEF, wall motion summary score, end-systolic volume (LVESV) and end-diastolic volume (LVEDV) from baseline to 90 days' post-initial infusion, and from day 90 (i.e., new baseline at beginning of cross-over phase) to day 180 (i.e., 90 days after second infusion). 6-minute walk test (6MWT), NYHA class, and Kansas City Cardiomyopathy Questionnaire (KCCQ) data were collected at days 30 and 90 days following initial and second infusions. Protocol pre-specified serum biomarkers (including NT pro B-type natriuretic peptide, troponin I, fibroblast growth factor 23 [FGF23], vascular endothelial growth factor [VEGF]), and lymphocyte proliferation panels (including levels of CD3, CD4, and natural killer [NK] cells,) were assessed at these same time points.

Due to the exploratory nature of this early phase study, a sample size of 23 patients was targeted as appropriate for a phase II-a primarily safety trial. Summary data are presented as mean or median as appropriate and all analyses used a two-sided 0.05 significance level. Comparative analyses for the primary safety endpoints of adverse and serious adverse events are presented in entirety and also by attributing adverse events to either the placebo or chronic itMSC therapy for the 90-day period following administration of either intervention. Secondary efficacy endpoints related to change from baseline were analyzed using the analysis of covariance, with treatment as a factor and the baseline value as a covariate, to determine least squares means (LSM), differences in LSMs compared with placebo, and 95% confidence intervals (CI). No imputations were performed for missing data. Data were analyzed using SAS version 9.3.

### Results

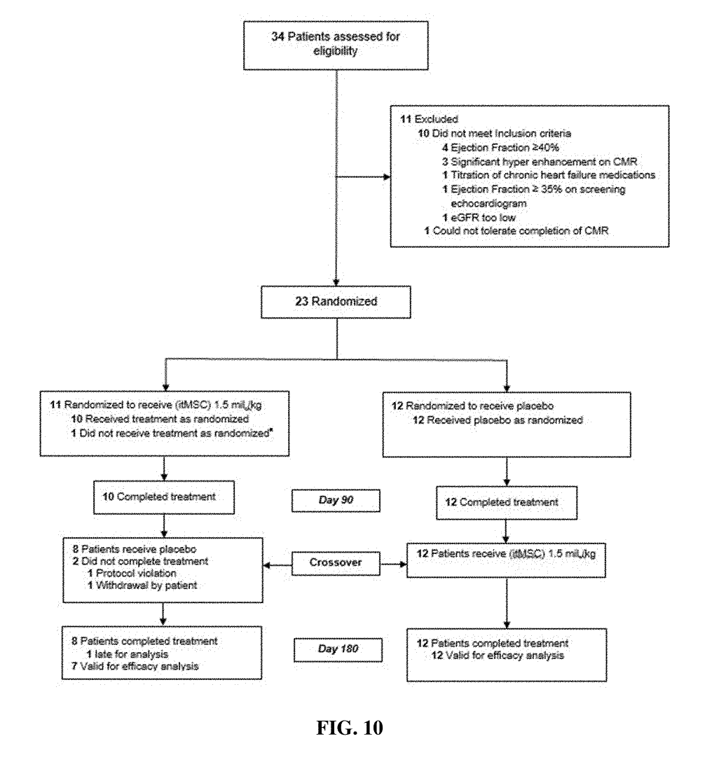

Of the 34 patients screened, 23 were randomized and 22 received the study intervention (FIG. 10). Patients were randomized from four sites across the United States. Overall, 12 patients randomized to placebo-chronic itMSC completed treatment at 90 days, and thus constituted the control group. The chronic itMSC group included 10 itMSC-placebo patients who completed study treatment at 90 days plus 12 placebo-chronic itMSC patients who completed study treatment at 180 days. Thus, the chronic itMSC group included 22 patients who completed treatment at 90 days following chronic itMSC infusion.

Baseline characteristics of the population are presented in Table 4. Mean age was 47.3 years and over two thirds of patients were white. All but 1 patient had NYHA class II symptoms and median NT-proBNP was significantly elevated at 212 pg/mL. Rates of comorbidities were generally low, with <25% of patients having hypertension, diabetes, atrial fibrillation and chronic kidney disease. At baseline, patients were receiving loop diuretics (72.7%), angiotensin-converting enzyme inhibitors/angiotensin II receptor blockers (100.0%), beta-blockers (100.0%), and mineralocorticoid receptor antagonists (81.8%).

TABLE 4
Baseline Characteristics of Study Population

| | All patients (n = 22)* | |
|---|---|---|
| Age, yrs | 47 · 3 | (12 · 8) |
| Male sex | 13 | (59 · 1%) |
| Weight, kg | 92 · 50 | (19 · 96) |
| Body mass index | 32 · 24 | (7 · 56) |
| Race | | |
| White | 15 | (68 · 2%) |
| Black | 7 | (31 · 8%) |
| Other | 0 | (0%) |
| NYHA class at baseline | | |
| Class II | 21 | (95 · 5%) |
| Class III | 1 | (4 · 5%) |
| Systolic blood pressure, mmHg | 120 | (17 · 1) |
| Diastolic blood pressure, mmHg | 74 | (9 · 6) |
| Heart rate, bpm | 78 | (14 · 2) |
| NT-proBNP, pg/mL | 212 | (IQR 841) |
| Troponin I, ug/mL | 0 · 009 | (0 · 003) |
| Serum sodium, mmol/L | 138 | (2) |
| Serum creatinine, mg/dL | 0 · 96 | (0 · 23) |
| AST, IU/L | 21 · 86 | (8 · 64) |
| ALT, IU/L | 21 · 82 | (9 · 57) |
| Total bilirubin, mg/dL | 0 · 80 | (0 · 40) |
| Albumin, g/dL | 4 · 2 | (0 · 36) |

Medical History

| | | |
|---|---|---|
| Hypertension | 5 | (22 · 7%) |
| Diabetes | 3 | (13 · 6%) |
| Atrial fibrillation | 2 | (9 · 1%) |
| Chronic kidney disease | 1 | (4 · 5%) |

Baseline Cardiac Magnetic Resonance Data

| | | |
|---|---|---|
| LVEF, % | 31.6 | (9.8) |
| LVESV, mL | 189.6 | (114.2) |
| LVEDV, mL | 264.9 | (120.4) |

Baseline Medications

| | | |
|---|---|---|
| Loop diuretic | 16 | (72 · 7%) |
| ACEI or ARB | 22 | (100 · 0%) |
| Mineralocorticoid Receptor | 18 | (81 · 8%) |
| Antagonist | | |
| Beta-blocker | 22 | (100 · 0%) |

*N reflects the randomized patients who received any study therapy Data reported as n (%) or mean (standard deviation) unless otherwise noted.
ACEI, angiotensin-converting enzyme inhibitor;
ALT, alanine transaminase;
ARB, angiotensin II receptor blocker;
AST, aspartate transaminase;
IQR, interquartile range;
LVEDV, left ventricular end-diastolic volume;
LVEF, left ventricular ejection fraction;
LVESV, left ventricular end-systolic volume;
NT-proBNP, N-terminal pro-B-type natriuretic peptide;
NYHA, New York Heart Association

Safety data are displayed in Table 5 and reflect events that occurred in the 90 days following placebo or chronic itMSC infusion. During the course of the study, no patients died. There was 1 hospitalization for atrial fibrillation within the placebo group. Rates of adverse and serious adverse events were generally balanced between the 2 groups. There were 2 cell related adverse effects, both local reactions near the area of intravenous infusion. Holter monitoring showed clinically significant abnormalities in 1 patient over the course of the study. This patient, randomized to the placebo-chronic itMSC group, had an event defined as “other” on day 60 and an episode of ventricular tachycardia on day 120.

TABLE 5
Adverse Events During the 90 Days Following
Each Study Intervention

| | Placebo (n = 22) | itMSC (n = 22) |
|---|---|---|
| Adverse events | 33 | 40 |
| Serious adverse events | 0 | 2 |
| Cell related adverse events* | 0 | 2 |
| All-cause hospitalization | 1 | 0 |
| All-cause death | 0 | 0 |

*Both infusion related; 1 episode of superficial thrombophlebitis; 1 episode of bruising at intravenous infusion site

### Cardiac Remodeling

TABLE 6
Change from Baseline in Cardiac Magnetic Resonance
Endpoints at 90 days

| | Difference of Means, | | P value |
|---|---|---|---|
| Endpoint | itMSC minus placebo (95% CI) | | (2-sided) |
| LV wall motion | 0 · 50 (−1 · 02-2 · 02) | | 0 · 52 |
| summary score | | | |
| LVEF (%) | 0 · 01 (−1 · 50-1 · 52) | | 0 · 99 |
| LVEDV (mL) | 1 · 67 (−8 · 60-11 · 93) | | 0 · 75 |
| LVESV (mL) | 0 · 67 (−7 · 28-8 · 62) | | 0 · 87 |
| Variable | Difference | 95% CI | P |

Initial Injection: itMSC (N = 10)

| | | | | |
|---|---|---|---|---|
| LVEF (%) | 2.31 | −0.09 | 4.71 | 0.06 |
| LVEDV (ml) | −17.86 | −35.03 | −0.69 | 0.04 |
| LVESV (ml) | −16.60 | −33.22 | 0.02 | 0.05 |

Initial Injection: Placebo (N = 12)

| | | | | |
|---|---|---|---|---|
| LVEF (%) | 1.62 | −0.82 | 4.05 | 0.17 |
| LVEDV (ml) | −10.56 | −30.54 | 9.43 | 0.27 |
| LVESV (ml) | −8.90 | −27.40 | 9.60 | 0.31 |

LV, left ventricular;
LVEDV, left ventricular end-diastolic volume;
LVEF, left ventricular ejection fraction;
LVESV, left ventricular end-systolic volume

### Health Status and Functional Capacity

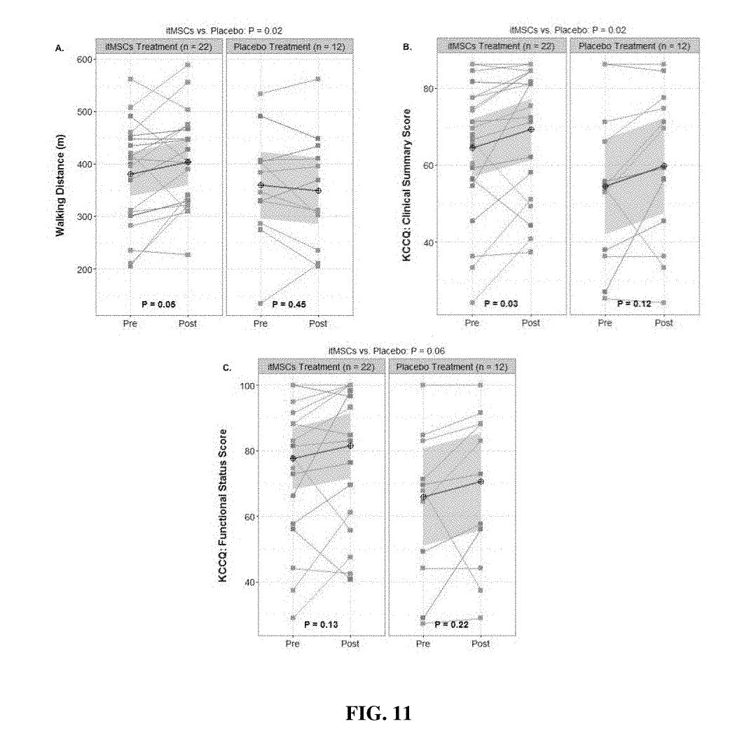

Treatment with chronic itMSCs resulted in statistically significant improvements in health status and functional capacity endpoints (Table 7). At 90 days following infusion, chronic itMSC therapy increased 6MWT distance an estimated 36.47 m (95% CI 5.98-66.97, p=0.02) more than placebo, representing a 15.9% greater change from baseline (FIG. 11A). Within the chronic itMSC phase, 6MWT distance increased 27.40 m (95% CI 0.28-54.52, p=0.05) compared to a nonsignificant decrease of 3.37 m (95% CI −28.46-21.72, p=0.79) during the placebo phase. Compared to placebo, chronic itMSC resulted in greater improvements in KCCQ clinical summary (+5.22, 95% CI 0.70-9.74, p=0.02) and functional status scores (+5.65, 95% CI −0.11-11.41, p=0.06) (FIGS. 11B and 11C). In addition, compared to placebo, chronic itMSC therapy was associated with a non-significant greater odds (OR 2.49, 95% CI 2.08-74.51, p=0.33) of lower NYHA classification at 90 days. Within the itMSC group, NYHA classification was statistically significant lower at 90 days (p=0.01), whereas there was no significant change in NYHA classification within the placebo group (p=0.19).

TABLE 7
Change from baseline in Health Status Endpoints at 90 days

| | | |
|---|---|---|
| | Difference of Means, | P value |
| Endpoint | itMSC minus placebo (95% CI) | (2-sided) |

6 Minute Walk Test

| | | |
|---|---|---|
| Distance (m) | 36 · 47 (5 · 98-66 · 97) | 0 · 02 |
| Distance (% change from | 15 · 94 (1 · 63-30 · 24) | 0 · 03 |
| baseline) | | |

Kansas City Cardiomyopathy Questionnaire

| Functional Status Score | 5 · 65 (−0 · 11-11 · 41) | 0 · 06 |
|---|---|---|
| Clinical Summary Score | 5 · 22 (0 · 70-9 · 74) | 0 · 02 |
| | Odds ratio (95% CI) [itMSC versus | |
| | placebo] | |

New York Heart Association Classification

| | | |
|---|---|---|
| Lower NYHA functional | 2 · 49 (0 · 40-15 · 34) | 0 · 33 |
| class at 90 days | | |

itMSC, ischemic tolerant mesenchymal stem cells;
NYHA, New York Heart Association

### Biomarker

In the chronic itMSC group, compared to baseline, there were significant changes in immunomodulatory markers at 30-days post-infusion, including an increase in the percentage of CD3 (p=0.045) and CD4 cells (p=0.006) and a decrease in absolute and percent NK cells (p=0.025 and p=0.006, respectively) (Table 8). Change from baseline in other biomarkers, including NT-proBNP and troponin I, during the itMSC phase were nonsignificant at 30 and 90 days' post-infusion.

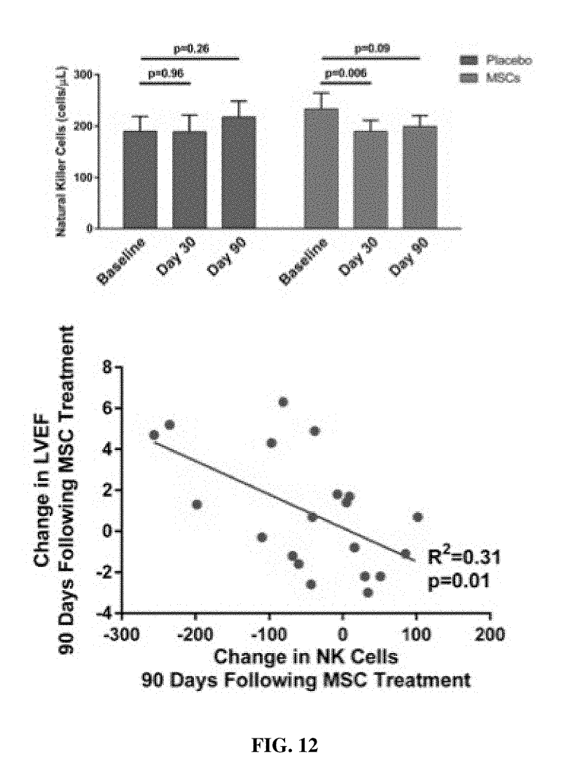

In ad hoc analysis, there was an inverse relationship between the degree of reduction in NK cells from baseline to 90 days and the magnitude of improvement in LVEF (p=0.01, R²=0.31) (FIG. 12) but not with LVEDV (p=0.29, R²=0.06) or LVESV (p=0.14, R²=0.12).

TABLE 8
Baseline and Change from Baseline During the itMSC Phase

| | | Mean | | P value |
|---|---|---|---|---|
| Biomarker | N | (standard deviation) | | (2-sided) |
| NT-proBNP (pg/mL) | | | | |
| Baseline | 22 | 806 · 27 | (1387 · 85) | — |
| Change from baseline to 30 d | 22 | −102 · 00 | (528 · 30) | 0 · 375 |
| Change from baseline to 90 d | 20 | 768 · 25 | (2945 · 53) | 0 · 258 |
| Troponin I (ng/mL) | | | | |
| Baseline | 22 | 0 · 009 | (0 · 003) | — |
| Change from baseline to 30 d | 22 | −0 · 001 | (0 · 004) | 0 · 083 |
| Change from baseline to 90 d | 20 | 0 · 000 | (0 · 006) | 1 · 00 |
| VEGF (pg/mL) | | | | |
| Baseline | 22 | 119 · 41 | (106 · 26) | — |
| Change from baseline to 30 d | 19 | −17 · 05 | (59 · 13) | 0 · 225 |
| Change from baseline to 90 d | 11 | 4 · 55 | (40 · 59) | 0 · 718 |
| FGF 23 (RU/mL) | | | | |
| Baseline | 21 | 187 · 57 | (248 · 50) | — |
| Change from baseline to 30 d | 19 | −7 · 47 | (110 · 57) | 0 · 772 |
| Change from baseline to 90 d | 11 | −58 · 55 | (281 · 58) | 0 · 506 |
| CD3 cells (Total T-cells), (%) | | | | |
| Baseline | 22 | 73 · 31 | (7 · 66) | — |
| Change from baseline to 30 d | 22 | 1 · 59 | (3 · 50) | 0 · 045 |
| Change from baseline to 90 d | 20 | 0 · 65 | (6 · 81) | 0 · 674 |
| CD4 cells (Helper T-cells), | | | | |
| (%) | | | | |
| Baseline | 22 | 49 · 45 | (7 · 28) | — |
| Change from baseline to 30 d | 22 | 2 · 09 | (3 · 18) | 0 · 006 |
| Change from baseline to 90 d | 20 | 1 · 30 | (6 · 73) | 0 · 399 |
| NK cells, (%) | | | | |
| Baseline | 22 | 10 · 45 | (4 · 92) | — |
| Change from baseline to 30 d | 22 | −1 · 32 | (2 · 57) | 0 · 025 |
| Change from baseline to 90 d | 20 | −1 · 05 | (4 · 32) | 0 · 090 |

FGF, fibroblast growth factor;
itMSC, ischemic tolerant mesenchymal stem cells;
NK, natural killer;
NT-proBNP, N-terminal pro-B-type natriuretic peptide;
VEGF, vascular endothelial growth factor

### DISCUSSION

This phase II-a trial was designed to evaluate the safety and preliminary efficacy of intravenous administration of chronic itMSCs in a relatively homogenous population of patients with non-ischemic cardiomyopathy. Overall, this study found single dose intravenous infusion of chronic itMSCs to be safe, well-tolerated, and to provide clinically relevant beneficial signals. Chronic itMSC therapy resulted in improvements in patient health status as demonstrated by KCCQ scores and 6MWT. 6MWT is strongly associated with peak exercise oxygen consumption, which is among the most powerful and objective predictors of functional status and short-term event free survival (Cahalin 1996).

Most prior stem cell studies have been designed under the assumption that the mechanism of benefit of stem cell therapy accrues only from activities derived from cells engrafted in the dysfunctional myocardium, whether these activities result in the cells differentiating into cardiac myocytes, stimulating resident cardiac stem cells to expand and form more functioning myocytes, or to broad paracrine and systemic activities that allow, for example, favorable cardiac remodeling, enhancement of angiogenesis, and decreased apoptosis (Kelkar 2015; Narita 2015). Thus, studies have focused on delivery methods that maximize myocardial engraftment, including intracoronary, transendocardial, or intramyocardial routes. However, such strategies involve catheter or surgical techniques and suffer major practical limitations in that a single injection of cells is unlikely to be curative and that multiple invasive cell administrations would likely be required over the course of the condition.

Because intravenous cell delivery results in low rates of cardiac engraftment, to our knowledge there has been only a single clinical trial assessing the efficacy of intravenous administration with that study limited to patients suffering acute myocardial infarction (Hare 2009). Interestingly, that study still operated under the belief that MSCs need robust engraftment in ischemic myocardium to show benefit and no data on potential systemic immunomodulatory effects of cell therapy were obtained.

In the present study, intravenous delivery of chronic itMSCs resulted in statistically significant alterations in several inflammatory cells, proving that the intravenous delivery of chronic itMSCs exert systemic effects—in particular on the inflammatory system. Importantly, we found a statistically significant inverse relationship between change in NK cell and LVEF, whereby the degree of NK cell reduction correlated with the magnitude of improvement in LVEF at 90 days (FIG. 12).

In conclusion, intravenous administration of itMSCs in patients with non-ischemic cardiomyopathy with reduced LVEF was safe and well-tolerated. At 90-days post-infusion, compared to placebo, a single dose of study therapy consistently improved multiple measurements of patient health status.

The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

### REFERENCES

Massie B M, O'Connor C M, Metra M, et al. Rolofylline, an adenosine A1-receptor antagonist, in acute heart failure. N Engl J Med 2010; 363:1419-1428.
Konstam M A, Gheorghiade M, Burnett J C Jr, et al. Effects of oral tolvaptan in patients hospitalized for worsening heart failure: the EVEREST Outcome Trial. JAMA 2007; 297:1319-1331.
Gheorghiade M, Bohm M, Greene S J, et al. Effect of aliskiren on postdischarge mortality and heart failure readmissions among patients hospitalized for heart failure: the ASTRONAUT randomized trial. JAMA 2013; 309:1125-1135.
Bayeva M, Gheorghiade M, Ardehali H. Mitochondria as a therapeutic target in heart failure. J Am Coll Cardiol 2013; 61:599-610.
Schelbert E B, Fonarow G C, Bonow R O, Butler J, Gheorghiade M. Therapeutic targets in heart failure: refocusing on the myocardial interstitium. J Am Coll Cardiol 2014; 63:2188-2198.
Wong T C, Piehler K M, Zareba K M, et al. Myocardial damage detected by late gadolinium enhancement cardiovascular magnetic resonance is associated with subsequent hospitalization for heart failure. J Am Heart Assoc 2013; 2:e000416.
Bolli R, Chugh A R, D'Amario D, et al. Cardiac stem cells in patients withischaemic cardiomyopathy (SCIPIO): initial results of a randomised phase 1 trial. Lancet 2011; 378:1847-1857.
Nowbar A N, Mielewczik M, Karavassilis M, et al. Discrepancies in autologous bone marrow stem cell trials and enhancement of ejection fraction (DAMASCENE): weighted regression and meta-analysis. Br Med J 2014; 348:g2688.
Sanganalmath S K, Bolli R. Cell therapy for heart failure: a comprehensive overview of experimental and clinical studies, current challenges, and future directions. Circ Res 2013; 113:810-834.
Vaduganathan M, Greene S J, Ambrosy A P, Gheorghiade M, Butler J. The disconnect between phase II and phase III trials of drugs for heart failure. Nat Rev Cardiol 2013; 10:85-97.
Greene S J, Gheorghiade M. Matching mechanism of death with mechanism of action: considerations for drug development for hospitalized heart failure. J Am Coll Cardiol 2014; 64:1599-1601.
Gheorghiade M, Pang P S, O'Connor C M, et al. Clinical development of pharmacologic agents for acute heart failure syndromes: a proposal for a mechanistic translational phase. Am Heart J 2011; 161:224-232.
Wagner A, Mahrholdt H, Holly T A, et al. Contrast-enhanced MRI and routine single photon emission computed tomography (SPECT) perfusion imaging for detection of subendocardial myocardial infarcts: an imaging study. Lancet 2003; 361:374-379.
Kim R J, Albert T S, Wible J H, et al. Performance of delayed-enhancement magnetic resonance imaging with gadoversetamide contrast for the detection and assessment of myocardial infarction: an international, multicenter, double-blinded, randomized trial. Circulation 2008; 117:629-637.
Kim R J, Wu E, Rafael A, et al. The use of contrast-enhanced magnetic resonance imaging to identify reversible myocardial dysfunction. N Engl J Med 2000; 343:1445-1453.
Ambrosy A P, Fonarow G C, Butler J, et al. The global health and economic burden of hospitalizations for heart failure: lessons learned from hospitalized heart failure registries. J Am Coll Cardiol 2014; 63(12): 1123-33.
Kelkar A A, Butler J, Schelbert E B, et al. Mechanisms Contributing to the Progression of Ischemic and Nonischemic Dilated Cardiomyopathy: Possible Modulating Effects of Paracrine Activities of Stem Cells. J Am Coll Cardiol 2015; 66(18): 2038-47.
Gheorghiade M, Larson C J, Shah S J, et al. Developing New Treatments for Heart Failure: Focus on the Heart. Circ Heart Fail 2016; [epub ahead of print] doi: 10.1161/CIRCHEARTFAILURE.115.002727
Sanganalmath S K, Bolli R. Cell therapy for heart failure: a comprehensive overview of experimental and clinical studies, current challenges, and future directions. Circ Res 2013; 113(6): 810-34.
Narita T, Suzuki K. Bone marrow-derived mesenchymal stem cells for the treatment of heart failure. Heart Fail Rev 2015; 20(1):53-68.
Bayeva M, Sawicki K T, Butler J, Gheorghiade M, Ardehali H. Molecular and cellular basis of viable dysfunctional myocardium. Circ Heart Fail 2014; 7(4): 680-91.
Cleland J G, Pennell D J, Ray S G, et al. Myocardial viability as a determinant of the ejection fraction response to carvedilol in patients with heart failure (CHRISTMAS trial): randomised controlled trial. Lancet 2003; 362(9377): 14-21.
Barsheshet A, Goldenberg I, Moss A J, et al. Response to preventive cardiac resynchronization therapy in patients with ischaemic and nonischaemic cardiomyopathy in MADIT-CRT. Eur Heart J 2011; 32(13): 1622-30.
Wilcox J E, Fonarow G C, Ardehali H, et al. “Targeting the Heart” in Heart Failure: Myocardial Recovery in Heart Failure With Reduced Ejection Fraction. JACC Heart Fail 2015; 3(9): 661-9.
Maslov L N, Podoksenov Iu K, Portnichenko A G, Naumova A V. [Hypoxic preconditioning of stem cells as a new approach to increase the efficacy of cell therapy for myocardial infarction]. Vestn Ross Akad Med Nauk 2013; (12): 16-25.
Kim R J, Wu E, Rafael A, et al. The use of contrast-enhanced magnetic resonance imaging to identify reversible myocardial dysfunction. N Engl J Med 2000; 343(20): 1445-53.
Cahalin L P, Mathier M A, Semigran M J, Dec G W, DiSalvo T G. The six-minute walk test predicts peak oxygen uptake and survival in patients with advanced heart failure. Chest 1996; 110(2): 325-32.
Hare J M, Traverse J H, Henry T D, et al. A randomized, double-blind, placebo-controlled, dose-escalation study of intravenous adult human mesenchymal stem cells (prochymal) after acute myocardial infarction. J Am Coll Cardiol 2009; 54(24): 2277-86.

1. A method of treating non-ischemic heart failure in a patient in need thereof, the method comprising administering to said patient mesenchymal stem cells, wherein administering said mesenchymal stem cells to said patient treats non-ischemic heart failure in said patient.

2. The method of claim 1, wherein said mesenchymal stem cells are administered to said patient systemically.

3. The method of claim 1, wherein said mesenchymal stem cells are administered to said patient intravenously.

4. The method of any one of claims 1-3, wherein said mesenchymal stem cells are ischemic tolerant mesenchymal stem cells.

5. The method of claim 4, wherein said mesenchymal stem cells are chronic ischemic tolerant mesenchymal stem cells.

6. The method of any one of claims 1-3, wherein said mesenchymal stem cells are exposed to low oxygen.

7. The method of any one of claims 1-3, wherein said mesenchymal stem cells are grown under low oxygen conditions.

8. The method of claim 7, wherein said mesenchymal stem cells are grown under low oxygen conditions for at least five passages.

9. The method of claim 7, wherein said mesenchymal stem cells are grown under low oxygen conditions for five passages.

10. The method of any one of claims 7-9, wherein said mesenchymal stem cells are grown exclusively under low oxygen conditions.

11. The method of any one of claims 1-10, wherein said mesenchymal stem cells are grown under low serum.

12. The method of any one of claims 1-11, wherein said mesenchymal stem cells are human cells.

13. The method of any one of claims 1-12, wherein said mesenchymal stem cells are allogeneic with respect to said patient.

14. The method of any one of claims 1-13, wherein said mesenchymal stem cells are obtained from one or more human donors that are between about 18 and 25 years old.

15. The method of any one of claims 1-14, wherein said mesenchymal stem cells are bone marrow mesenchymal stem cells.

16. The method of any one of claims 1-15, wherein said mesenchymal stem cells are administered in a composition that is free of cells selected from the group consisting of: CD19+ cells (B cells); CD45+ cells (T cells); and CD 14+ cells (macrophages/dendritic cells); and a combination thereof.

17. The method of any one of claims 1-15, wherein said mesenchymal stem cells are purified.

18. The method of any one of claims 1-17, wherein said mesenchymal stem cells comprises less than about 2% HLA-DR+ cells.

19. The method of any one of claims 1-18, wherein said mesenchymal stem cells express CD105, CD73 and CD90.

20. The method of any one of claims 1-19, wherein said patient has NYHA class II or NYHA class III heart failure.

21. The method of any one of claims 1-20, wherein said patient has dysfunctional viable myocardium without scarring.

22. The method of any one of claims 1-21, wherein said patient has a reduced left ventricular ejection fraction (LVEF).

23. The method of any one of claims 1-21, wherein said patient has an LVEF of less than about 40 percent.

24. The method of any one of claims 1-23, wherein said patient is a human.

25. The method of any one of claims 1-24, wherein said mesenchymal stem cells are administered to said patient at a dose of about 1.5×10⁶ mesenchymal stem cells/kg.

26. The method of any one of claims 1-25, wherein administering said mesenchymal stem cells to said patient improves cardiac function in said patient compared to said patient's cardiac function prior to administering said mesenchymal stem cells.

27. The method of claim 26, wherein said improvement in said cardiac function is an improvement in said patient's Kansas City Cardiomyopathy Questionnaire (KCCQ) score compared to said patient's KCCQ score prior to administering said mesenchymal stem cells.

28. The method of any one of claims 1-27, wherein administering said mesenchymal stem cells to said patient improves six minute walk performance in said patient compared to said patient's six-minute walk performance prior to administering said mesenchymal stem cells.

29. The method of any one of claims 1-28, wherein administering said mesenchymal stem cells reduces NK cells in said patient compared to NK cell levels in said patient prior to administering said mesenchymal stem cells.

30. A method of improving cardiac function in a patient in need thereof, the method comprising administering to said patient mesenchymal stem cells, wherein administering said mesenchymal stem cells to said patient improves cardiac function in said patient.

31. The method of claim 30, wherein said mesenchymal stem cells are administered to said patient systemically.

32. The method of claim 30, wherein said mesenchymal stem cells are administered to said patient intravenously.

33. The method of any one of claims 30-32, wherein said mesenchymal stem cells are ischemic tolerant mesenchymal stem cells.

34. The method of claim 33, wherein said mesenchymal stem cells are chronic ischemic tolerant mesenchymal stem cells.

35. The method of any one of claims 30-32, wherein said mesenchymal stem cells are exposed to low oxygen.

36. The method of any one of claims 30-32, wherein said mesenchymal stem cells are grown under low oxygen conditions.

37. The method of claim 36, wherein said mesenchymal stem cells are grown under low oxygen conditions for at least five passages.

38. The method of claim 36, wherein said mesenchymal stem cells are grown under low oxygen conditions for five passages.

39. The method of any one of claims 36-38, wherein said mesenchymal stem cells are grown exclusively under low oxygen conditions.

40. The method of any one of claims 30-39, wherein said mesenchymal stem cells are grown under low serum.

41. The method of any one of claims 30-40, wherein said mesenchymal stem cells are human cells.

42. The method of any one of claims 30-41, wherein said mesenchymal stem cells are allogeneic with respect to said patient.

43. The method of any one of claims 30-42, wherein said mesenchymal stem cells are obtained from one or more human donors that are between about 18 and 25 years old.

44. The method of any one of claims 30-43, wherein said mesenchymal stem cells are bone marrow mesenchymal stem cells.

45. The method of any one of claims 30-44, wherein said mesenchymal stem cells are administered in a composition that is free of cells selected from the group consisting of: CD19+ cells (B cells); CD45+ cells (T cells); CD 14+ cells (macrophages/dendritic cells); and a combination thereof.

46. The method of any one of claims 30-45, wherein said mesenchymal stem cells are purified.

47. The method of any one of claims 30-46, wherein mesenchymal stem cells comprise less than about 2% HLA-DR+ cells.

48. The method of any one of claims 30-47, wherein said mesenchymal stem cells express CD105, CD73 and CD90.

49. The method of any one of claims 30-48, wherein said patient has non-ischemic heart failure.

50. The method of any one of claims 30-49, wherein said patient has NYHA class II or NYHA class III heart failure.

51. The method of any one of claims 30-50, wherein said patient has dysfunctional viable myocardium without scarring.

52. The method of any one of claims 30-51, wherein said patient has a reduced left ventricular ejection fraction (LVEF).

53. The method of any one of claims 30-52, wherein said patient has an LVEF of less than about 40 percent.

54. The method of any one of claims 30-53, wherein said patient is a human.

55. The method of any one of claims 30-54, wherein said mesenchymal stem cells are administered to said patient at a dose of about 1.5×10⁶ mesenchymal stem cells/kg.

56. The method of any one of claims 30-55, wherein administering said mesenchymal stem cells to said patient increases LVEF in said patient compared to said patient's LVEF prior to administering said mesenchymal stem cells.

57. The method of any one of claims 30-56, wherein administering said mesenchymal stem cells to said patient improves said patient's Kansas City Cardiomyopathy Questionnaire (KCCQ) score compared to said patient's KCCQ score prior to administering said mesenchymal stem cells.

58. The method of any one of claims 30-57, wherein administering said mesenchymal stem cells to said patient improves six minute walk performance in said patient compared to said patient's six-minute walk performance prior to administering said mesenchymal stem cells.

59. The method of any one of claims 30-58, wherein administering said mesenchymal stem cells reduces NK cells in said patient compared to NK cell levels in said patient prior to administering said mesenchymal stem cells.
